(19) United States
(12) Patent Application Publication
(10) Pub. No.: US 2004/0122422 A1
(43) Pub. Date: Jun. 24, 2004

(54) Medical device on helical support

(76) Inventor: Moshe Ein-Gal, Ramat Hasharon (IL)

Correspondence Address: Dekel Patent Ltd., Beit HaRofim, 18 Menuha VeNahala Street, Room 27, Rehovot, IL

(21) Appl. No.: 10/327,028

(22) Filed: Dec. 24, 2002

(51) Int. Cl. A61B 18/18

(52) U.S. Cl. 606/46; 604/20

(57) ABSTRACT

Apparatus comprising a helical support adapted to corkscrew into a tissue, and a medical device assembled with the helical support, the medical device being adapted to release a substance, or in another embodiment, to emit non-RF energy. In yet another embodiment, the medical device may comprise a non-helical radio frequency (RF) electrode mounted on a distal tip of the helical support.

### FIELD OF THE INVENTION

The present invention relates generally to medical devices mounted on a helical support, such as but not limited to, electrosurgical electrodes for radio frequency (RF) tissue ablation or devices for administration of therapeutic agents into tissues.

### BACKGROUND OF THE INVENTION

Radio frequency (RF) tissue ablation is a well-known technique for making thermal lesions in the vicinity of an uninsulated tip of an electrode due to tissue coagulation caused by resistive heating. The electrode can be applied directly on superficial structures, surgically, endoscopically, laparascopically, or even via a transcatheter access such as a treatment for symptomatic cardiac arrhythmias. If the electrode is formed as a needle, then the electrode may be inserted interstitially, and guided by imaging.

A general problem in RF tissue ablation is limitation in lesion size. Increasing the power to the electrode or exposure time to the tissue increases the amount of energy delivered around the electrode and thereby increases the lesion size. However, at high temperatures (e.g., above 100° C.) at the electrode-tissue interface, the impedance increases significantly because of desiccation and tissue adhesion followed by charring around the electrode tip. This leads to an abrupt fall in lesion current (and delivered effect) and no further energy is delivered around the electrode, and no further tissue heating occurs. This phenomenon tends to limit lesion size in the transverse direction around the electrode. The longitudinal dimension of the lesion is basically dependent on the length of the uninsulated part of the electrode.

Attempts have been made in the prior art to overcome the abovementioned problems. One known solution is that of a helical electrode. The helical electrode provides an enlarged surface area as compared to relatively straight or needle-like electrodes. For example, U.S. Pat. No. 6,497,704 to Ein-Gal describes different kinds of helical electrode arrangements. In one embodiment, the electrodes may be configured as a pair of bipolar concentric (sharing a common center) or eccentric (off-center) helices. A plurality of electrodes may be mounted on the same helical insulated support. A central insulated rod may be added to helical electrodes for motion stabilization.

The helical electrode assembly may coagulate a cylindrical envelope of tissue, while at the same time sparing a cylinder of tissue at the center of the helix. For example, the helical arrangement may be used to coagulate prostate tissue around the urethra without causing coagulation of the urethra itself. In other treatment plans, it may be desirable to cause necrosis of the inner cylindrical volume of the helical electrode assembly. In such a case, the helical electrode assembly may coagulate the tissue surrounding the inner cylindrical volume in such a way such that the blood supply to the inner non-coagulated cylindrical tissue is cut off. The non-coagulated cylindrical tissue may then be left to die due to the absence of a sufficient blood supply from the coagulated cylindrical envelope, thereby increasing the amount of tissue that undergoes necrosis and shortening treatment time.

### SUMMARY OF THE INVENTION

The present invention seeks to provide novel medical devices mounted on a helical support. As opposed to the prior art, in the present invention, the helical support is not necessarily an electrode, but instead may be non-conducting. For example, in one embodiment of the invention, assorted medical devices may be mounted on a distal tip of a non-conducting helical support. Examples of such medical devices may be, but are not limited to, a straight electrode for RF tissue ablation, an enclosure from which a therapeutic agent, chemical or other substance may be released, or a miniature electromagnetic wave module for emitting infrared, light, ultraviolet, microwave, X-ray or gamma ray energy, as is described more in detail hereinbelow.

There is thus provided in accordance with an embodiment of the present invention helical apparatus comprising a helical support adapted to corkscrew into a tissue, and a medical device assembled with the helical support, the medical device comprising a substance disposed in and releasable from an enclosure.

In accordance with an embodiment of the present invention an actuator may be in communication with the medical device and operative to actuate the medical device to release the substance from the enclosure. The actuator may be connected to the medical device by a connection passing through the helical support and/or a communication link that does not helically pass through the helical support.

Further in accordance with an embodiment of the present invention the enclosure may comprise a permeable membrane through which the substance passes. For example, the substance may pass through apertures formed in the membrane or may pass through the membrane by an osmotic process. Additionally or alternatively in accordance with an embodiment of the present invention, the membrane may be adapted for reverse osmosis for drawing matter into the enclosure.

Still further in accordance with an embodiment of the present invention the substance may pass through the membrane by iontophoresis.

In accordance with an embodiment of the present invention the enclosure may comprise a rupturable membrane, wherein the actuator ruptures the membrane in order to release the substance.

Further in accordance with an embodiment of the present invention the enclosure may comprise a membrane disintegrable in a presence of a body fluid, wherein the substance is released from the enclosure upon sufficient disintegration of the membrane.

In accordance with an embodiment of the present invention a manipulator may be in operable connection with the helical support and adapted to move the helical support.

Further in accordance with an embodiment of the present invention a plurality of the helical supports may be provided, wherein a helical pitch of one of the helical supports is shifted axially with respect to a helical pitch of another of the helical supports.

Still further in accordance with an embodiment of the present invention a sensor may be assembled with at least one of the helical support and the medical device.

There is also provided in accordance with an embodiment of the present invention helical apparatus comprising a helical support adapted to corkscrew into a tissue with an energy module operative to emit non-RF energy. The energy module may comprise an optical energy source, infrared energy source, ultraviolet energy source, microwave energy source, X-ray energy source and/or gamma ray energy source.

In accordance with an embodiment of the present invention the energy module may comprise an acoustic module operative to emit acoustic energy.

There is also provided in accordance with an embodiment of the present invention helical apparatus comprising a non-electrically conducting helical support adapted to corkscrew into a tissue, and a medical device mounted on a distal tip of the helical support, the medical device comprising a non-helical radio frequency (RF) electrode. The RF electrode may be energized by an actuator, e.g., an external electrode.

### BRIEF DESCRIPTION OF THE DRAWINGS

The present invention will be understood and appreciated more fully from the following detailed description, taken in conjunction with the drawings in which:

FIG. 1 is a simplified illustration of helical apparatus constructed and operative in accordance with an embodiment of the present invention;

FIG. 2 is a simplified, enlarged illustration of an enclosure of the helical apparatus of FIG. 1, wherein the enclosure comprises a membrane formed with apertures;

FIG. 3 is a simplified, enlarged illustration of an enclosure of the helical apparatus of FIG. 1, wherein the enclosure comprises an osmotic membrane;

FIG. 4 is a simplified, enlarged illustration of an enclosure of the helical apparatus of FIG. 1, wherein the enclosure comprises a membrane disintegrable in a presence of a body fluid;

FIG. 5 is a simplified, enlarged illustration of an enclosure of the helical apparatus of FIG. 1, wherein the enclosure comprises a rupturable membrane;

FIG. 6 is a simplified, enlarged illustration of an enclosure of the helical apparatus of FIG. 1, wherein a substance may pass through a membrane by iontophoresis;

FIG. 7 is a simplified illustration of helical apparatus, constructed and operative in accordance with another embodiment of the present invention; and

FIG. 8 is a simplified illustration of helical apparatus, constructed and operative in accordance with still another embodiment of the present invention.

### DETAILED DESCRIPTION OF PREFERRED EMBODIMENTS

Reference is now made to FIG. 1, which illustrates helical apparatus 10, constructed and operative in accordance with an embodiment of the present invention.

Helical apparatus 10 may comprise a helical support 12 adapted to corkscrew into a tissue (not shown). A medical device 14 may be assembled with helical support 12, such as but not limited to, at a distal tip 15 of support 12 or at any other portion in or on support 12. In some embodiments of the invention, medical device 14 is not a radio frequency (RF) electrode, but instead may be a medical device capable of releasing a substance and/or a quantity of non-electrode energy, examples of which are now described more in detail.

In accordance with one embodiment of the invention, medical device 14 comprises a substance 16 disposed in and releasable from an enclosure 18. Substance 16 may be, without limitation, a drug, therapeutic agent, analgesic, relaxant, anesthetic agent, tracer or dye, just to name some.

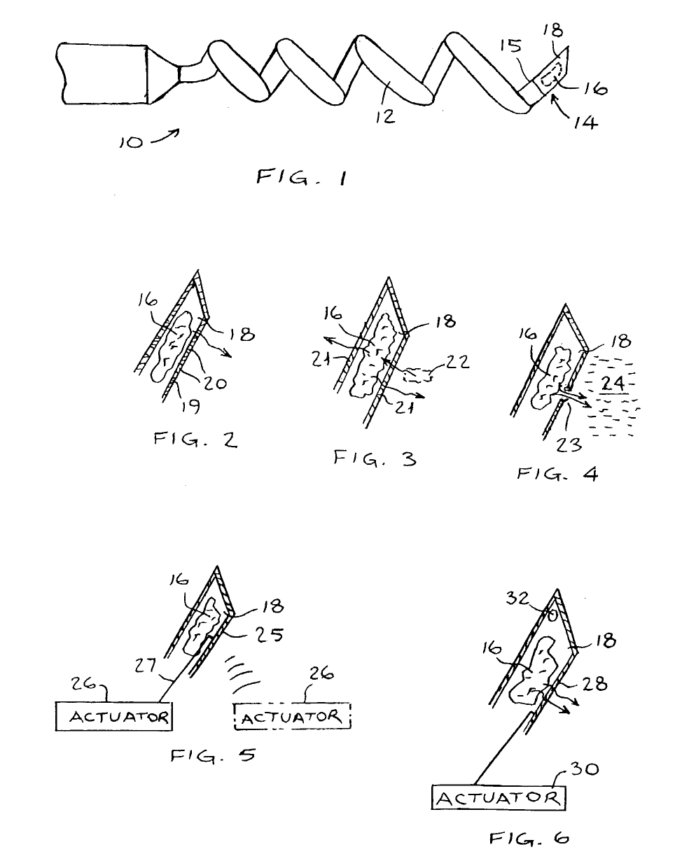

The enclosure 18 may comprise a permeable membrane through which substance 16 passes. For example, as seen in the enlarged view of FIG. 2, enclosure 18 may comprise a membrane 19 formed with apertures 20, such as but not limited to holes or pores, and substance 16 may pass through apertures 20. In another example, as seen in the enlarged view of FIG. 3, enclosure 18 may comprise an osmotic membrane 21 and substance 16 passes through membrane 21 by an osmotic process. In such an embodiment, membrane 21 may be optionally adapted for reverse osmosis for drawing matter 22 (e.g., body fluids) into enclosure 18.

In yet another example, as seen in the enlarged view of FIG. 4, enclosure 18 comprises a membrane 23 disintegrable in a presence of a body fluid 24, such as but not limited to a biodegradable membrane that degrades in the presence of blood, lymph or gastrointestinal fluid, for example. The substance 16 may be released from enclosure 18 upon sufficient disintegration of membrane 23.

In still another example, as seen in the enlarged view of FIG. 5, enclosure 18 may comprises a rupturable membrane 25. An actuator 26 may be in communication with medical device 14 for actuating medical device 14, which in this embodiment means rupturing membrane 25 in order to release substance 16 from enclosure 18. Actuator 26 may be connected to medical device 14 by a connection passing through helical support 12, such as but not limited to, a wire 27 attached to rupturable membrane 25. Sufficient proximal pulling or distal pushing of wire 27 may rupture membrane 25 and release substance 16 from enclosure 18. Alternatively, actuator 26 may be in communication with medical device 14 by a communication link that does not helically pass through helical support 12. For example, membrane 25 may be rupturable upon application of an external electrical signal, in which case, actuator 26 may be a wireless remote control that transmits the required electrical signal.

In a further example, as seen in the enlarged view of FIG. 6, substance 16 may pass through a membrane 28 by iontophoresis. In such an embodiment, an actuator 30 may be provided which is either in wired communication with membrane 28 (e.g., a wire passing through helical support 12) or in wireless communication with membrane 28 (e.g., an infrared link or a BLUETOOTH link). Actuator 30 may comprise a constant electromotive force source of sufficient strength to drive ions of substance 16 into a body fluid or tissue (which acts as an electrolyte) adjacent membrane 28. An example of an iontophoresis application is in the prevention of bacterial infection associated with certain medical procedures. Heavy metal ions such as gold, silver, platinum, iron, and copper have been demonstrated to have antibacterial activity.

In any of the embodiments of the invention, described hereinabove or hereinbelow, a sensor 32 may be placed in or on helical apparatus 10, such as at distal tip 15 thereof or in or on medical device 14, an example of which is shown in FIG. 6. For example, sensor 32 may comprise a temperature sensor (e.g., thermocouple), pressure sensor, or biosensor (e.g., level of oxygen in the blood) and may be used in a closed loop feedback control system to control operation of helical apparatus 10.

In any of the embodiments of the invention, described hereinabove or hereinbelow, the helical support may comprise a generally hollow lumen for passage therethrough of a fluid, e.g., a liquid or gel electrolyte, for example.

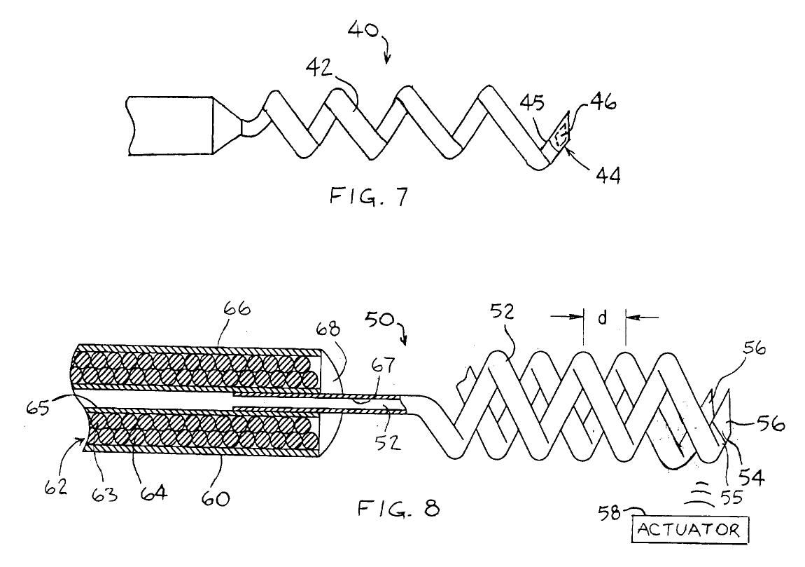

Reference is now made to FIG. 7, which illustrates helical apparatus 40, constructed and operative in accordance with another embodiment of the present invention.

Helical apparatus 40 may comprise a helical support 42 adapted to corkscrew into a tissue (not shown). A medical device 44 may be assembled with helical support 42, such as but not limited to, at a distal tip 45 of support 42 or at any other portion in or on support 42. In accordance with one embodiment of the invention, medical device 44 comprises an energy module 46 operative to emit non-RF energy. For example, energy module 46 may comprise, without limitation, an optical energy source (e.g., a laser), infrared energy source, ultraviolet energy source, microwave energy source, X-ray energy source and/or gamma ray energy source. Alternatively, energy module 46 may comprise an acoustic module operative to emit acoustic (e.g., ultrasonic) energy. Energy module 46 may be self-contained or may be activated or energized by an external actuator (not shown), in wired or wireless connection. In the case of a laser, fiber optic connections may be provided to the energy module 46.

Reference is now made to FIG. 8, which illustrates helical apparatus 50, constructed and operative in accordance with still another embodiment of the present invention.

Helical apparatus 50 may comprise one or more non-electrically conducting helical supports 52 adapted to corkscrew into a tissue (not shown). A medical device 54 may be mounted on a distal tip 55 of helical support 52, wherein medical device 54 comprises a non-helical radio frequency (RF) electrode 56. In this embodiment, helical support 52 is sufficiently non-conducting that it does not serve as an electrode. Rather electrode 56 is carried on and introduced into the tissue by helical support 52, and only the non-helical electrode 56 acts as the electrode. Helical apparatus 50 may comprise a pair of electrodes 56 configured to operate as bipolar electrodes. Alternatively, a single electrode 56 may be used as a monopolar electrode energized by an actuator 58, e.g., an external electrode.

In any of the embodiments of the invention, as shown exemplary in FIG. 8, a manipulator 60 may be in operable connection with the helical support(s) 52 and adapted to move the helical support(s) 52, e.g., to screw each helical support 52 into the tissue, wherein helical support 52 cuts a helical path into the tissue. One way of transferring torque to helical support 52 is by means of a torque cable 62. Torque cable 62 may include two coils 63 and 64, which are wound in opposite directions about a tube 65 housed in an insulating catheter sleeve 66. Such a torque cable is commercially available from Lake Region Manufacturing Company of Chaska, Minn., USA. A proximal portion 67 of helical support 52 may be tightly fit into tube 65 through an end cap 68 of sleeve 66.

Manipulator 60 may be electrically, pneumatically, hydraulically or mechanically driven, and may include without limitation, a servomotor, step motor, linear actuator, rotary actuator, vibrator or solenoid, for example. Manipulator 60 may be coupled to a plurality of the helical supports 52 so as to move all the helical supports 52 generally simultaneously in synchronization with one another. Alternatively, manipulator 60 may be coupled to a single helical support 52 for independent movement and control of the helical supports 52.

In one embodiment of the invention, a helical pitch of one of the helical supports 52 may be shifted axially, such as by a distance d, with respect to the helical pitch of another of the helical supports 52. The distance d may one one-half pitch, for example. Helical supports 52 with uniform or different pitches may be used. The helical supports 52 may be configured as a pair of bipolar concentric (sharing a common center) or eccentric (off-center) helices.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. Helical apparatus comprising:
a helical support adapted to corkscrew into a tissue; and
a medical device assembled with said helical support, said medical device comprising a substance disposed in and releasable from an enclosure.

2. The apparatus according to claim 1, further comprising an actuator in communication with said medical device and operative to actuate said medical device to release said substance from said enclosure.

3. The apparatus according to claim 2, wherein said actuator is connected to said medical device by a connection passing through said helical support.

4. The apparatus according to claim 2, wherein said actuator is in communication with said medical device by a communication link that does not helically pass through said helical support.

5. The apparatus according to claim 1, wherein said enclosure comprises a permeable membrane through which said substance passes.

6. The apparatus according to claim 5, wherein said substance passes through apertures formed in said membrane.

7. The apparatus according to claim 5, wherein said substance passes through said membrane by an osmotic process.

8. The apparatus according to claim 7, wherein said membrane is adapted for reverse osmosis for drawing matter into said enclosure.

9. The apparatus according to claim 5, wherein said substance passes through said membrane by iontophoresis.

10. The apparatus according to claim 2, wherein said enclosure comprises a rupturable membrane and said actuator is operative to rupture said membrane in order to release said substance.

11. The apparatus according to claim 1, wherein said enclosure comprises a membrane disintegrable in a presence of a body fluid, wherein said substance is released from said enclosure upon sufficient disintegration of said membrane.

12. The apparatus according to claim 1, further comprising a manipulator in operable connection with said helical support and adapted to move said helical support.

13. The apparatus according to claim 1, further comprising a plurality of said helical supports.

14. The apparatus according to claim 13, wherein a helical pitch of one of said helical supports is shifted axially with respect to a helical pitch of another of said helical supports.

15. The apparatus according to claim 1, further comprising a sensor assembled with at least one of said helical support and said medical device.

16. Helical apparatus comprising:
a helical support adapted to corkscrew into a tissue, said helical support comprising an energy module operative to emit non-RF energy.

17. The apparatus according to claim 16, wherein said energy module comprises at least one of an optical energy source, infrared energy source, ultraviolet energy source, microwave energy source, X-ray energy source and gamma ray energy source.

18. The apparatus according to claim 16, wherein said energy module comprises an acoustic module operative to emit acoustic energy.

19. Helical apparatus comprising:
a non-electrically conducting helical support adapted to corkscrew into a tissue; and
a medical device mounted on a distal tip of said helical support, said medical device comprising a non-helical radio frequency (RF) electrode.

20. The apparatus according to claim 19, further comprising an actuator in communication with said medical device and operative to energize said RF electrode.
